(19) United States
(12) Patent Application Publication
(10) Pub. No.: US 2023/0227611 A1
(43) Pub. Date: Jul. 20, 2023

(54) PLASMA POLYMERIZED THIN FILM AND PREPARING METHOD THEREOF

(71) Applicant: RESEARCH & BUSINESS FOUNDATION SUNGKYUNKWAN UNIVERSITY, Suwon-si (KR)

(72) Inventors: Donggeun JUNG, Seoul (KR); Namwuk BAEK, Suwon-si (KR); Jihwan CHA, Suwon-si (KR); Taesoon JANG, Bucheon-si (KR); Shinwon KANG, Bucheon-si (KR); Yoonsu PARK, Gwangmyeong-si (KR); Hyuna LIM, Hwaseong-si (KR)

(73) Assignee: RESEARCH & BUSINESS FOUNDATION SUNGKYUNKWAN UNIVERSITY, Suwon-si (KR)

(21) Appl. No.: 18/098,996

(22) Filed: Jan. 19, 2023

(30) Foreign Application Priority Data
Jan. 20, 2022 (KR) 10-2022-0008644
Oct. 26, 2022 (KR) 10-2022-0138864

(51) Int. Cl. C08G 77/12; C08G 77/06; C08J 5/18; C12N 5/0775; C12N 5/09; B05D 1/00; B05D 3/14; B05D 3/02

(52) U.S. Cl. CPC C08G 77/12; C08G 77/06; C08J 5/18; C12N 5/0662; C12N 5/0693; B05D 1/62; B05D 3/145; B05D 3/0254; C08J 2383/05; C12N 2533/30

(57) ABSTRACT

The present application relates to a plasma polymer thin film and a method for preparing the same, the plasma polymer thin film prepared using a first precursor material represented by the following Chemical Formula 1:

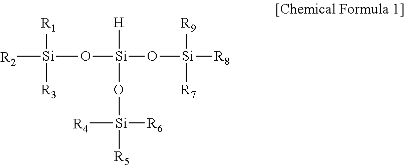

(In Chemical Formula 1,

R₁ to R₉ are each independently H or a C₁-C₅ substituted or unsubstituted alkyl group, and when R₁ to R₉ are substituted, the substituent is an amino group, a hydroxyl group, a cyano group, a halogen group, a nitro group, or a methoxy group).

### CROSS-REFERENCE TO RELATED APPLICATIONS

This application claims the priority of Korean Patent Application No.10-2022-0008644 (filed on Jan. 20, 2022) and No.10-2022-0138864 (filed on Oct. 26, 2022), in the Korean Intellectual Property Office, the disclosure of which is incorporated herein by reference.

### BACKGROUND

### Field

The present application relates to a plasma polymer thin film and a method for preparing the same.

### Description of the Related Art

One of the critical steps in modern semiconductor fabrication is a step of forming a metal and dielectric thin film on a substrate by chemical reaction of gases. Such a deposition process is referred to as chemical deposition or chemical vapor deposition (CVD). In a typical thermal CVD process, a reactive gas is provided to the surface of a substrate, and a heat-induced chemical reaction occurs on the substrate surface to form a predetermined thin film. The thermal CVD process is performed at high temperatures, and the structure of a device having a layer formed on the substrate may be damaged due to high temperatures. One of the methods capable of solving such a problem, that is, depositing a metal and dielectric thin film at a relatively low temperature, is a plasma enhanced CVD (PECVD) method.

The plasma enhanced CVD method applies radio frequency (RF) energy to a reaction region and thus promotes excitation and/or dissociation of reactive gases, thereby generating a plasma of highly reactive species. Due to the high reactivity of such species, the energy required for the chemical reaction to occur is reduced, and thus the temperature required for thin film formation may be lowered in the plasma enhanced CVD process. Due to the introduction of such apparatuses and methods, there are many cases of enabling preparing processes that can significantly reduce the size of semiconductor devices.

Meanwhile, silicon dioxide (SiO₂) or silicon oxyfluoride (SiOF), which has been mainly used as an interlayer insulating film so far, has problems such as high resistance capacitance delay (RC delay) when preparing ultra-high integrated circuits of 0.5 μm or less. Therefore, in order to reduce the resistance capacitance delay (RC delay) of a multilayer metal film used in an integrated circuit of a semiconductor device, research on forming the interlayer insulating film used in the metal wiring with a material having a low dielectric constant (relative dielectric constant k<4.0) has actively been conducted lately. Such a low-k thin film may be formed of an inorganic material, such as a SiCOH film mixed with Si, O, C, H, and the like, and an amorphous carbon (a-C:F) film doped with fluorine, or an organic material containing carbon (C).

Low-dielectric materials currently being considered as substitutes for SiO₂ include organic polymers such as benzocyclobutene (BCB), SILK (Supplier: Dow Chemical), fluorinated poly(arylene ether) (FLARE) (Supplier: Allied Signals), polyimide, etc., which are mainly used for spin coating, and porous thin film materials such as Black Diamond (Supplier: Applied Materials), Coral (Supplier: Novellus), and xerogel or aerogel, which are formed by chemical vapor deposition.

Here, since pores having a size of several nanometers are formed in a thin film of a material having a low dielectric constant formed by a spin casting method in which curing is performed after spin coating, the thin film density is reduced to form a dielectric having a low dielectric constant. Generally, the organic polymers deposited by spin coating have advantages such as a low dielectric constant and excellent flatness on the whole, but are unsuitable for semiconductor applications since their heat resistance limit temperature is lower than 450° C. so that their thermal stability is poor. Since the size of pores is particularly large and the pores are not uniformly distributed in the thin film, the mechanical strength of the thin film is low, resulting in various difficulties during device preparing. In addition, there are problems such as poor adhesion to upper and lower wiring materials, occurrence of high stress due to thermal curing peculiar to organic polymer thin films, and reduced reliability of the device due to a change in dielectric constant due to adsorption of ambient moisture.

Korean Patent Nos. 10-1506801 and 10-2138102, which are the background technology of the present application, relate to a low-k plasma polymer thin film and a method for preparing the same. The above patents disclose a low-k plasma polymer thin film prepared using cross-shaped and H-shaped organic/inorganic precursor materials, respectively, and a method for preparing the same, but there has been a difficulty in maintaining a satisfactory level of dielectric constant value due to the low deposition rate of the thin film and the limitation of changing the low dielectric constant value after plasma exposure. Since the low-k thin film is unavoidably exposed to plasma during a subsequent process in a semiconductor process, the width of a change in characteristics after plasma exposure should be small.

Therefore, the inventors of the present application have achieved an improved deposition rate compared to the conventional art by preparing a plasma polymer thin film using a new T-type organic/inorganic precursor material. In addition, the conventional plasma polymer shows a change in characteristics after exposure to plasma in a subsequent process due to its low density and low carbon ratio, but the plasma polymer thin film according to the present disclosure shows a small change in characteristics after exposure to plasma due to its appropriate carbon content and density.

### SUMMARY

The present application is to solve the above-mentioned problems of the conventional art, and aims to provide a plasma polymer thin film and a method for preparing the same.

However, the technical problem to be achieved by the embodiments of the present application is not limited to the technical problems as described above, and other technical problems may exist.

As a technical means for achieving the above-described technical problem, a first aspect of the present application provides a plasma polymer thin film prepared using a first precursor material represented by the following Chemical Formula 1:

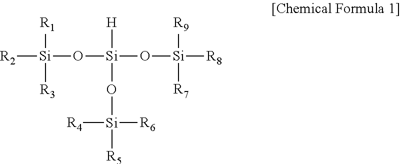

(in Chemical Formula 1,

R₁ to R₉ are each independently H or a C₁-C₅ substituted or unsubstituted alkyl group, and when R₁ to R₉ are substituted, the substituent is an amino group, a hydroxyl group, a cyano group, a halogen group, a nitro group, or a methoxy group).

According to one embodiment of the present application, the plasma polymer thin film may be prepared by using a second precursor material, which is a hydrocarbon of a liquid state at 25° C. and 1 atm, together with the first precursor material, but is not limited thereto.

According to one embodiment of the present application, the second precursor material may include C₆-C₁₂ alkane, alkene, cycloalkane, or cycloalkene, but is not limited thereto.

According to one embodiment of the present application, the second precursor material may include cyclohexane, but is not limited thereto.

According to one embodiment of the present application, the first precursor material may have a T-shaped structure, but is not limited thereto.

According to one embodiment of the present application, the plasma polymer thin film may be prepared using a plasma enhanced CVD (PECVD) method, but is not limited thereto.

A second aspect of the present application provides a method for preparing a plasma polymer thin film, in which a plasma-polymerized thin film is deposited on a substrate using a first precursor material represented by the following Chemical Formula 1:

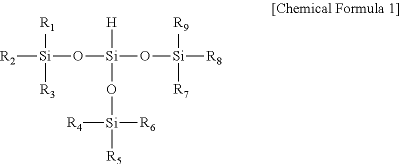

(in Chemical Formula 1,

R₁ to R₉ are each independently H or a C₁C₅ substituted or unsubstituted alkyl group, and when R₁ to R₉ are substituted, the substituent is an amino group, a hydroxyl group, a cyano group, a halogen group, a nitro group, or a methoxy group).

According to one embodiment of the present application, the step of depositing the plasma-polymerized thin film on a substrate may be to deposit a plasma-polymerized thin film on a substrate by using a second precursor material, which is a hydrocarbon of the liquid state at 25° C. and 1 atm, together with the first precursor material, but is not limited thereto.

According to one embodiment of the present application, the second precursor material may include C₆-C₁₂ alkane, alkene, cycloalkane, or cycloalkene, but is not limited thereto.

According to one embodiment of the present application, the second precursor material may include cyclohexane, but is not limited thereto.

According to one embodiment of the present application, the method may further include a step of post-treating the thin film deposited on the substrate, but is not limited thereto.

According to one embodiment of the present application, the post-treatment may be performed by a process selected from the group consisting of inductively coupled plasma (ICP) treatment, rapid thermal annealing (RTA), and a combination thereof, but is not limited thereto.

According to one embodiment of the present application, the step of depositing the plasma-polymerized thin film on a substrate may include steps of: evaporating the first precursor material and the second precursor material in a bubbler; inflowing the evaporated precursor materials from the bubbler to flow them into a reactor for plasma deposition; and forming a plasma-polymerized thin film on a substrate within the reactor using plasma of the reactor, but is not limited thereto.

According to one embodiment of the present application, the reactor may contain a carrier gas selected from the group consisting of argon (Ar), helium (He), neon (Ne), and combinations thereof, but is not limited thereto.

According to one embodiment of the present application, the reactor may have a carrier gas pressure of 1×10⁻¹ Torr to 100×10⁻¹ Torr, but is not limited thereto.

According to one embodiment of the present application, the substrate within the reactor may have a temperature of 20° C. to 50° C., but is not limited thereto.

According to one embodiment of the present application, the reactor may have a power supplied thereto of 10 W to 90 W, but is not limited thereto.

A third aspect of the present application provides a stem cell culture substrate including the plasma polymer thin film according to the first aspect of the present application.

According to one embodiment of the present application, the stem cells may have a spheroid shape, but are not limited thereto.

The above-described problem solving means is merely exemplary and should not be construed as intended to limit the present application. In addition to the exemplary embodiments described above, additional embodiments may exist in the drawings and detailed description of the invention.

According to the above-mentioned problem solving means of the present application, the plasma polymer thin film according to the present application can prepare a plasma polymer thin film having a lower dielectric constant value while satisfying the mechanical strength required in an existing semiconductor process by using a precursor material having a T-shaped structure structurally different from the precursor material used in conventional plasma polymers.

Further, the plasma polymer thin film according to the present application has a small change width in characteristics after plasma exposure due to appropriate carbon content and density, and thus may have characteristics more suitable for use in the semiconductor process than conventional thin films.

The plasma polymer thin film according to the present application, due to its characteristics of being thermally stable and having a very low dielectric constant, can replace a dielectric used in the metal multilayer wiring of the semiconductor device, and can improve the resistance capacitance delay (RC delay) that increases according to the refinement of the metal multilayer wiring.

Particularly, the plasma polymer thin film according to the present application reduces a dielectric constant while maintaining characteristics required in the semiconductor process so that it can be directly applied to a metal multilayer wiring process and can improve the above-described resistance capacitance delay (RC delay).

However, the effects obtainable from the present application are not limited to the effects described above, and other effects may exist.

### BRIEF DESCRIPTION OF THE DRAWINGS

FIG. 1 is a schematic diagram of a plasma enhanced chemical vapor deposition (CVD) apparatus used in preparing a plasma polymer thin film according to one embodiment of the present application;

FIG. 2 is a schematic diagram of a rapid thermal annealing (RTA) apparatus used in preparing a plasma polymer thin film according to one embodiment of the present application;

FIG. 3 is a graph showing relative dielectric constant values of a plasma polymer thin film deposited using a first precursor material prepared according to one embodiment of the present application;

FIG. 4 is a graph showing hardnesses of a plasma polymer thin film deposited using a first precursor material prepared according to one embodiment of the present application;

FIG. 5 is a graph showing elastic moduli (moduli) of a plasma polymer thin film deposited using a first precursor material prepared according to one embodiment of the present application;

FIG. 6 is a graph showing chemical structures obtained by Fourier infrared spectroscopy in a plasma polymer thin film deposited using a first precursor material prepared according to one embodiment of the present application;

FIG. 7 is a graph showing deposition rates of plasma polymer thin films prepared according to an Example and Comparative Examples of the present application;

FIG. 8 is a graph showing relative dielectric constant values of plasma polymer thin films prepared according to an Example and Comparative Examples of the present application;

FIG. 9 is a graph showing relative dielectric constant values before and after heat treatment of plasma polymer thin films prepared according to an Example and Comparative Examples of the present application;

FIG. 10 is a graph comparing hardnesses of plasma polymer thin films prepared according to an Example and Comparative Examples of the present application;

FIG. 11 is a graph comparing elastic moduli of plasma polymer thin films prepared according to an Example and Comparative Examples of the present application;

FIG. 12 is a graph showing dielectric constant change values before and after plasma exposure of plasma polymer thin films prepared according to an Example and Comparative Examples of the present application;

FIG. 13 is a graph showing dielectric constant values according to elastic moduli and carbon ratios of plasma polymer thin films prepared according to an Example and Comparative Examples of the present application; and

FIG. 14 is results of culturing cells in a plasma polymer thin film according to one embodiment of the present application.

### DETAILED DESCRIPTION OF THE EMBODIMENT

Hereinafter, embodiments of the present application will be described in detail with reference to the accompanying drawings so that those with ordinary skill in the art to which the present application pertains will easily be able to implement the present application. However, the present application may be implemented in various different forms and is not limited to the embodiments described herein. Further, parts irrelevant to the description are omitted in order to clearly describe the present application in the drawings, and similar reference numerals are attached to similar parts throughout the specification.

In the whole specification of the present application, when a part is said to be “connected” with another part, it not only includes a case that the part is “directly connected” to the other part, but also includes a case that the part is “electrically connected” to the other part with another element being interposed therebetween.

In the whole specification of the present application, when any member is positioned “on”, “over”, “above”, “beneath”, “under”, and “below” another member, this not only includes a case that the any member is brought into contact with the other member, but also includes a case that another member exists between two members.

In the whole specification of the present application, if a prescribed part “includes” a prescribed element, this means that another element can be further included instead of excluding other elements unless any particularly opposite description exists.

When unique prepare and material allowable errors of numerical values are suggested to mentioned meanings of terms of degrees used in the present specification such as “about”, “substantially”, etc., the terms of degrees are used in the numerical values or as a meaning near the numerical values, and the terms of degrees are used to prevent that an unscrupulous infringer unfairly uses a disclosure content in which exact or absolute numerical values are mentioned to help understanding of the present application. Further, in the whole specification of the present application, “a step to do ˜” or “a step of ˜” does not mean “a step for ˜”.

In the whole specification of the present application, a term of “a combination thereof” included in a Markush type expression, which means a mixture or combination of one or more selected from the group consisting of constituent elements described in the Markush type expression, means including one or more selected from the group consisting of the constituent elements.

In the whole specification of the present application, description of “A and/or B” means “A, B, or A and B”.

Hereinafter, a plasma polymer thin film according to the present application and a method for preparing the same will be described in detail with reference to embodiments and examples and drawings. However, the present application is not limited to these embodiments and examples and drawings.

As a technical means for achieving the above-mentioned technical problem, a first aspect of the present application provides a plasma polymer thin film prepared using a first precursor material represented by the following Chemical Formula 1:

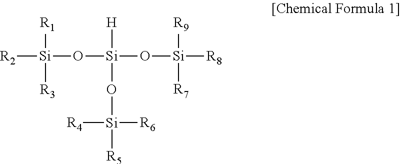

(In Chemical Formula 1,

R₁ to R₉ are each independently H or a C₁-C₅ substituted or unsubstituted alkyl group, and when R₁ to R₉ are substituted, the substituent is an amino group, a hydroxyl group, a cyano group, a halogen group, a nitro group, or a methoxy group).

For example, the first precursor may be a compound represented by the following Chemical Formula 2, but is not limited thereto:

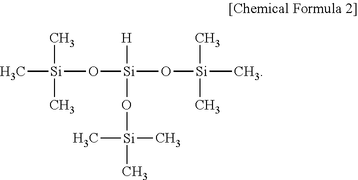 [SMILES: C[Si](C)(C)O[SiH](O[Si](C)(C)C)O[Si](C)(C)C]

The plasma polymer thin film according to the present application may form a plasma polymer thin film having a lower dielectric constant value while satisfying mechanical strength required in the existing semiconductor process by forming nanopores within the plasma polymer thin film using a first precursor material having a T-shaped structure structurally different from the precursor material used in conventional plasma polymers. For example, it may form a plasma polymer thin film having the dielectric constant value (k) of less than 4.0, but is not limited thereto.

Furthermore, the plasma polymer thin film according to the present application is thermally stable using the first precursor material, and due to characteristics of having a very low dielectric constant, can replace the dielectric used in the metal multilayer wiring of the semiconductor device and can improve resistance capacitance delay (RC delay), which increases according to refinement of the metal multilayer wiring.

Further, the plasma polymer thin film according to the present application has a small change width in characteristics after plasma exposure due to appropriate carbon content and density, and thus may have characteristics more suitable for use in the semiconductor process than conventional thin films.

According to one embodiment of the present application, it may be prepared by using a second precursor material, which is a hydrocarbon of a liquid state at 25° C. and 1 atm, together with the first precursor material, but is not limited thereto. Meanwhile, the plasma polymer thin film may be prepared using only the first precursor material without the second precursor material.

The second precursor material may be a hydrocarbon of a liquid state at 25° C. and 1 atm. When the second precursor material is a hydrocarbon, it may exhibit good bonding force with the first precursor material, and it can be advantageous in that it is easy to form a plasma polymer thin film, and furthermore, the mechanical strength and elasticity of the thin film can be improved due to the presence of a large number of C—Hₓ bond structures. Furthermore, the second precursor material is particularly advantageous in that it is in a liquid state in a standard state (25° C. and 1 atm) as described above to be applied to a bubbler of a plasma enhanced CVD (PECVD) apparatus to be described later. In general, a substance that is in a liquid state in a standard state is easily applied to a bubbler, stability of thin film deposition is excellent compared to in a solid state, a larger amount can be stored than in a gaseous state, and furthermore hydrocarbons in liquid state may be preferred in the role (evaporating the precursor) of the bubbler.

According to one embodiment of the present application, the second precursor material may include C₆-C₁₂ alkane, alkene, cycloalkane, or cycloalkene, but is not limited thereto. If the carbon number of the second precursor material is less than C₆, it is difficult to be in a liquid state in the standard state, the molecular weight is small so that there may be a problem in that thin film deposition is not easy due to a decrease in mutual bonding force with the first precursor material. Meanwhile, if the carbon number of the second precursor material is larger than C₁₂, it may be in a solid state in the standard state and may have a problem in that it is difficult to evaporate in a bubbler for deposition.

According to one embodiment of the present application, the second precursor material may include cyclohexane, but is not limited thereto.

The first precursor material and the second precursor material may be used in combination at the same time, and as described above, due to their chemical and structural characteristics, mutual bonding can be easily achieved, and the stability of the thin film is increased so that a plasma polymer thin film having improved mechanical properties while having a low dielectric constant value may be provided.

According to one embodiment of the present application, the first precursor material may have a T-shaped structure, but is not limited thereto. It may be possible to form a plasma polymer thin film having a lower dielectric constant value than the plasma polymer thin film prepared using the conventional precursor while satisfying the mechanical strength required in the existing semiconductor process by forming nanopores within the plasma polymer thin film using a first precursor material having a T-shaped structure.

According to one embodiment of the present application, the plasma polymer thin film may be prepared using a plasma enhanced chemical vapor deposition (hereinafter referred to as ‘PECVD’) method, but is not limited thereto. In the plasma enhanced CVD method as described above, plasma of highly reactive species is generated so that the first precursor material and the second precursor material may be effectively decomposed or excited to perform various chemical reactions, and for example, they may be combined and polymerized to form a plasma polymer thin film. In the case of such a polymer thin film, pores having a size of nanometers or less may be formed so that it may have relatively excellent mechanical strength while having a low dielectric constant.

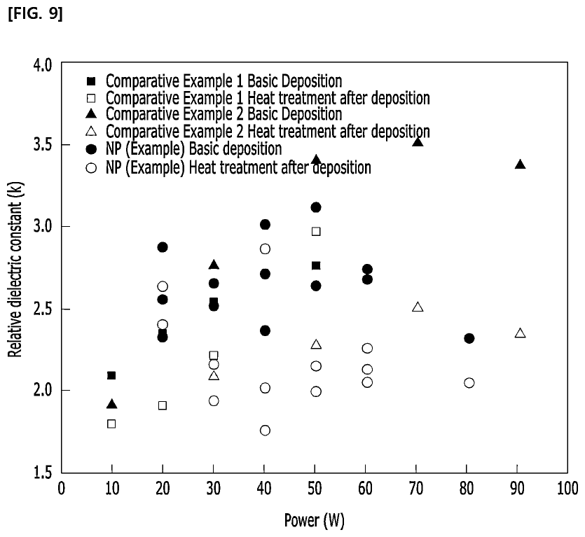

When depositing a thin film using the plasma enhanced CVD method, a thin film having a composition in which the first precursor material and the second precursor material are present at a predetermined ratio may be formed by inputting the first precursor material and the second precursor material at a predetermined ratio. Furthermore, the input amount or input ratio of the first precursor material and the second precursor material may be determined by adjusting the temperature of the bubbler and/or the flow rate of carrier gases such as argon (Ar). For example, when a first bubbler is 40° C. and a second bubbler is 25° C., a thin film may be deposited by maintaining the flow rate ratio of a first carrier gas to a second carrier gas corresponding to the input ratio of the first precursor material and the second precursor material at a ratio of 1:1 to 1:5. When the flow rate ratio of the second carrier gas is greater than 5 times that of the first carrier gas, SiOₓ in the thin film is significantly reduced, making it difficult to use it as an interlayer insulating film, and when the flow rate ratio of the second carrier gas does not reach 1 times that of the first carrier gas, it may be difficult for the second precursor component to sufficiently flow into the thin film. Furthermore, the plasma polymer thin film may be deposited using a plasma enhanced CVD method and then post-treated using RTA. It was confirmed that the dielectric constant of the plasma polymer thin film according to the present disclosure may be significantly reduced by performing the post-treatment (see FIG. 9).

The second aspect of the present application provides a method for preparing a plasma polymer thin film, in which a plasma-polymerized thin film is deposited on a substrate using a first precursor material represented by the following Chemical Formula 1:

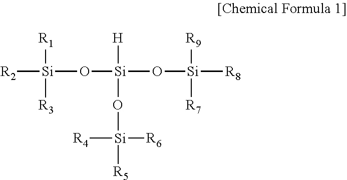

(In Chemical Formula 1,

R₁ to R₉ are each independently H or a C₁-C₅ substituted or unsubstituted alkyl group, and when R₁ to R₉ are substituted, the substituent is an amino group, a hydroxyl group, a cyano group, a halogen group, a nitro group, or a methoxy group).

With respect to the method for preparing a plasma polymer thin film according to the second aspect of the present application, detailed descriptions of parts overlapping with the first aspect of the present application have been omitted, but even if the descriptions have been omitted, the contents described in the first aspect of the present application may be equally applied to the second aspect of the present application.

The first precursor may be represented by the following Chemical Formula 2, but is not limited thereto:

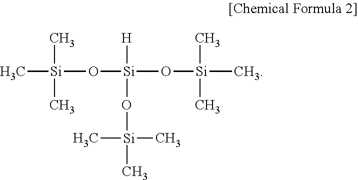 [SMILES: C[Si](C)(C)O[SiH](O[Si](C)(C)C)O[Si](C)(C)C]

Preferably, the first precursor may include tris(trimethylsiloxy)silane.

The plasma polymer thin film prepared according to the above-mentioned preparing method may form a plasma polymer thin film having a lower dielectric constant value while satisfying mechanical strength required in the existing semiconductor process by forming nanopores within the plasma polymer thin film during preparing of the thin film using a first precursor material having a T-shaped structure structurally different from the precursor material used in conventional plasma polymers.

Furthermore, the plasma polymer thin film prepared according to the above-mentioned preparing method is thermally stable using the first precursor material, and due to characteristics of having a very low dielectric constant, can replace a dielectric used in a metal multilayer wiring of a semiconductor device and can improve resistance capacitance delay (RC delay), which increases according to refinement of the metal multilayer wiring.

Further, the plasma polymer thin film according to the present application has a small change width in characteristics after plasma exposure due to appropriate carbon content and density, and thus may have characteristics more suitable for use in the semiconductor process than conventional thin films.

According to one embodiment of the present application, the step of depositing the plasma-polymerized thin film on a substrate may be to deposit a plasma-polymerized thin film on a substrate using a second precursor material, which is a hydrocarbon of a liquid state at 25° C. and 1 atm, together with the first precursor material, but is not limited thereto. Meanwhile, the plasma polymer thin film may be prepared using only the first precursor material without the second precursor material.

The second precursor material may be a hydrocarbon of a liquid state at 25° C. and 1 atm. When the second precursor material is a hydrocarbon, it may exhibit good bonding force with the first precursor material, and it can be advantageous in that it is easy to form a plasma polymer thin film, and furthermore, the mechanical strength and elasticity of the thin film can be improved due to the presence of a large number of C—Hₓ bond structures. Furthermore, the second precursor material is particularly advantageous in that it is in a liquid state in a standard state (25° C. and 1 atm) as described above when it is applied to a bubbler of a plasma enhanced CVD (PECVD) apparatus to be described later. In general, a substance that is in a liquid state in a standard state is easily applied to a bubbler, stability of thin film deposition is excellent compared to in a solid state, a larger amount can be stored than in a gaseous state, and furthermore hydrocarbons in liquid state may be preferred in the role (evaporating the precursor) of the bubbler.

According to one embodiment of the present application, the second precursor material may include C₆-C₁₂ alkane, alkene, cycloalkane, or cycloalkene, but is not limited thereto. If the carbon number of the second precursor material is less than C₆, it is difficult for the second precursor material to be in a liquid state in the standard state, the molecular weight thereof is small so that there may be a problem in that thin film deposition is not easy due to a decrease in mutual bonding force with the first precursor material. Meanwhile, if the carbon number of the second precursor material is larger than C₁₂, the second precursor material may be in a solid state in the standard state, and there may be a problem in that it is difficult to evaporate the second precursor material in a bubbler for deposition.

According to one embodiment of the present application, the second precursor material may include cyclohexane, but is not limited thereto.

The first precursor material and the second precursor material may be used in combination at the same time, and as described above, due to their chemical and structural characteristics, mutual bonding may be easily achieved, and the stability of the thin film is increased so that a plasma polymer thin film having improved mechanical properties while having a low dielectric constant value may be provided.

According to one embodiment of the present application, the method may further include a step of post-treating the thin film deposited on the substrate, but is not limited thereto.

According to one embodiment of the present application, the post-treatment may be performed by a process selected from the group consisting of inductively coupled plasma (hereinafter referred to as ‘ICP’) treatment, rapid thermal annealing (hereinafter referred to as ‘RTA’), and a combination thereof, but is not limited thereto. The dielectric constant may be improved by also using a method of lowering the dielectric constant by forming pores within the dielectric through the post-treatment. At this time, since the T-shaped Si—O bond of the first precursor is more solid than other bonds, a solid polymer thin film may be formed using the first precursor material. The post-treatment step may not be performed, or may be performed using one or both of the two treatment methods.

Hereinafter, a method for preparing a plasma polymer thin film according to one embodiment of the present disclosure will be described in more detail with reference to the accompanying drawings.

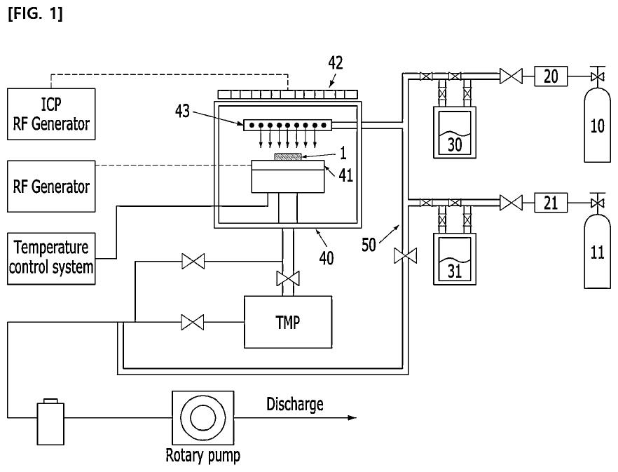

FIG. 1 is a schematic diagram of a plasma enhanced CVD (hereinafter referred to as ‘PECVD’) apparatus used in preparing a plasma polymer thin film according to one embodiment of the present disclosure.

As shown in FIG. 1, the PECVD apparatus used in preparing a plasma polymer thin film according to one embodiment of the present disclosure includes first and second carrier gas storage units 10 and 11 containing carrier gases such as argon (Ar), first and second flow controllers 20 and 21 that control the number of moles of gases passing through the first and second carrier gas storage units 10 and 11, first and second bubblers 30 and 31 containing a solid or liquid precursor, and a reactor 40 defining a predetermined reaction region. The carrier gas storage units 10 and 11, the flow controllers 20 and 21, the bubblers 30 and 31, and the reactor 40 are connected via a transport pipe 50. The reactor 40 is configured by including an RF electrode 41 located under a substrate 1, an ICP RF coil 42 located above the reactor 40, and a showerhead 43 having a plurality of openings allowing the gases to be uniformly introduced thereinto. An exhaust system is provided at the bottom of the reactor 40 to discharge various materials remaining inside the reactor 40 to the outside during the deposition reaction or after the reaction is completed.

According to the above description, a method of depositing a thin film using a PECVD apparatus is as follows.

According to one embodiment of the present application, the step of depositing the plasma-polymerized thin film on a substrate may include steps of: evaporating the first precursor material and the second precursor material in a bubbler; inflowing the evaporated precursor material from the bubbler and flowing it into a reactor for plasma deposition; and forming a plasma-polymerized thin film on a substrate in the reactor using plasma of the reactor, but is not limited thereto.

The first precursor material and the second precursor material are each contained in the first and second bubblers 30 and 31, and the first and second bubblers 30 and 31 are heated to a temperature sufficient to evaporate each precursor material. Here, it does not matter which of the two bubblers 30 and 31 contains each precursor material, but the heating temperature of each bubbler may be adjusted according to the type of precursor contained in the bubbler.

Each of the first and second carrier gas storage units 10 and 11 may contain argon (Ar), helium (He), neon (Ne), or a combined gas thereof as a carrier gas, and it may flow through the transport pipe 50 by the first and second flow controllers 20 and 21. The carrier gas moving along the transport pipe 50 is flown into the precursor solution of the bubblers 30 and 31 through the bubbler inflow pipe, generates bubbles, carries the vapor phase precursor, and enters again the transport pipe 50 through the bubbler discharge pipe. In this case, the ratio of the first and second precursor materials inputted into the reactor 40 may be adjusted by adjusting the flow rates of the first and second carrier gases.

More specifically, when the first bubbler is 40° C. and the second bubbler is 25° C., the flow rate ratio of the first carrier gas to the second carrier gas may be adjusted to a ratio of 1:1 to 1:5 so that the first and second precursor materials are inputted into the reactor, but is not particularly limited thereto. The carrier gas and the evaporated precursor passing through the bubblers 30 and 31 and flowing along the transport pipe 50 are injected through the showerhead 43 of the reactor 40. At this time, the RF electrode 41 activates the carrier gas and precursor injected through the showerhead 43. After injecting the carrier gas and precursor through the showerhead 43 of the reactor 40, the activated precursor is deposited on a substrate 1 to become a thin film. After the deposition reaction is completed, the remaining gas is discharged to the outside by an exhaust system provided at the bottom of the reactor 40.

According to one embodiment of the present application, the reactor may contain a carrier gas selected from the group consisting of argon (Ar), helium (He), neon (Ne), and combinations thereof, but is not limited thereto. Preferably, the reactor may contain argon as a carrier gas.

According to one embodiment of the present application, the reactor may have a carrier gas pressure of 1×10⁻¹ Torr to 100×10⁻¹ Torr, but is not limited thereto.

According to one embodiment of the present application, the temperature of the substrate in the reactor may be 20° C. to 50° C., but is not limited thereto. If the temperature of the substrate is out of this range, there may be a difficulty in forming a thin film with appropriate properties. When the temperature of the substrate is 200° C., the deposition rate of the thin film tends to decrease, and furthermore, a higher substrate temperature may inhibit C—Hₓ formation in the thin film and cause the formation of SiO₂.

According to one embodiment of the present application, the power supplied to the reactor may be 10 W to 90 W, but is not limited thereto. If the power is higher or lower than this, there may be a problem in that a thin film having a low dielectric constant with desired properties is not formed.

The pressure of such a carrier gas, the temperature of the substrate 1, the supply power, etc. are for activating the precursor material to form an optimal range of plasma that can be deposited on the substrate 1, and can be appropriately adjusted by those skilled in the art depending on the type of precursor material.

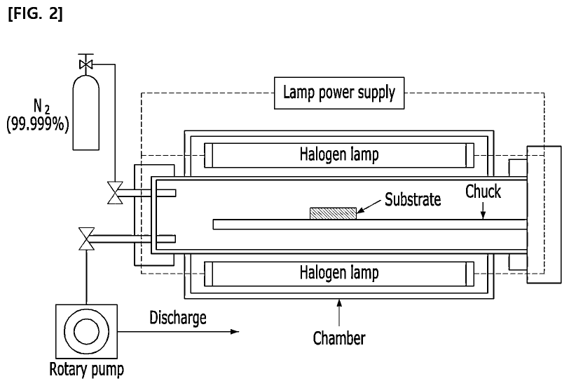

FIG. 2 is a schematic diagram of an RTA apparatus used in preparing a plasma polymer thin film according to one embodiment of the present application.

The RTA apparatus is used to activate electrons during heat treatment of specimens and semiconductor device process, and to change the interface between thin films or between the wafer and the thin film. In addition, the RTA apparatus may also be used to transform the state of a grown thin film and reduce loss due to ion implantation. Such RTA is implemented with a heated halogen lamp and a hot chuck. Unlike a furnace, RTA has a short process duration, and thus it is also called Rapid Thermal Process (RTP). Post-treatment may be performed on the plasma-deposited thin film in the previous step using such a heat treatment apparatus.

The inside of the RTA apparatus is surrounded by a plurality of halogen lamps positioned around it, and the lamps generate heat while emitting orange light. Such an RTA apparatus can heat-treat the thin film, which has undergone plasma deposition in the previous step, and the substrate 1 on which the thin film is placed at 300° C. to 600° C. At this time, if the temperature during the post-treatment is less than 300° C., the properties of the initially deposited thin film do not change, and if the temperature exceeds 600° C., there may be a problem in that the structure of the thin film structurally changes from a low dielectric constant thin film to a SiO₂ thin film. More preferably, it may be preferable to rapidly increase the temperature from the initial temperature to the above temperature within 5 minutes and perform the heat treatment for 1 to 5 minutes in that the thin film structure can be effectively changed. RTA post-treatment may be performed under a pressure of 1×10⁻¹ Torr to 100×10⁻¹ Torr using nitrogen gas.

The third aspect of the present application provides a stem cell culture substrate including the plasma polymer thin film according to the first aspect of the present application.

With respect to the stem cell culture substrate according to the third aspect of the present application, detailed descriptions of parts overlapping with the first aspect and/or the second aspect of the present application have been omitted, but even if the descriptions have been omitted, the contents described in the first aspect and/or the second aspect of the present application may be equally applied to the third aspect of the present application.

According to one embodiment of the present application, the stem cells may have a spheroid shape, but are not limited thereto.

Hereinafter, the present disclosure will be described in more detail through Examples, but the following Examples are for illustrative purposes only and are not intended to limit the scope of the present application.

[Example] Preparing of Plasma Polymer Thin Film (ppNP)

A precursor solution was vaporized using the PECVD apparatus shown in FIG. 1 by putting tris(trimethylsiloxy)silane (hereinafter referred to as NP) as a first precursor material into a first bubbler 30 and heating it to 65° C. after placing a silicon wafer on an RF electrode 41 in a reactor 40 and creating a vacuum state of the 10⁻² Torr region. 99.999% ultra-high purity argon (Ar) gas was used as a carrier gas. The argon gas supplied and injected the first precursor material to the showerhead 43 of the reactor 40 via the transport pipe 50 by passing through the bubbler 30 and deposited the first precursor material on the substrate 1 by plasma. At this time, AC power of 13.56 Hz and 90 W or less was used to generate the plasma, and plasma polymerization was performed at a pressure of 1.0 Torr or less and a temperature of 200° C. or less. The plasma polymer thin film thus deposited will be referred to as ‘ppNP’.

Furthermore, copolymerization was performed through the above preparing method by selecting cyclohexane (hereinafter referred to as ‘CHex’) as a second precursor material in the preparation of the plasma polymer. Such a copolymerized plasma polymer thin film will be referred to as ‘ppNP:CHex’.

The ppNP thin film was post-treated using the RTA apparatus shown in FIG. 2. The ppNP thin film was placed on a chuck, and heat was generated with 12 halogen lamps surrounding the ppNP thin film to heat-treat the ppNP thin film at 500° C. for 5 minutes in a nitrogen atmosphere. The nitrogen gas had a pressure of 1.0 Torr.

### COMPARATIVE EXAMPLE 1

### Preparing of Plasma Polymer Thin Film (ppTTMSS)

A plasma polymer thin film was prepared in the same manner as in Example except that tetrakis(trimethylsilyloxy)silane (hereinafter referred to as TTMSS) was used as a first precursor material.

Specifically, Comparative Example 1 is a plasma polymer thin film prepared using a cross-shaped precursor material disclosed in Korean Patent No. 10-1506801.

### COMPARATIVE EXAMPLE 2

### Preparing of Plasma Polymer Thin Film (ppOMBTSTS)

A plasma polymer thin film was prepared in the same manner as in Example except that 1,1,1,3,5,7,7,7-octamethyl-3,5-bis(trimethylsiloxy)tetrasiloxane (hereinafter referred to as OMBTSTS) was used as the first precursor material.

Specifically, Comparative Example 2 is a plasma polymer thin film prepared using the H-shaped precursor material disclosed in Korean Patent No. 10-2138102.

### EXPERIMENTAL EXAMPLE 1

### Characteristic Analysis Experiment of ppNP

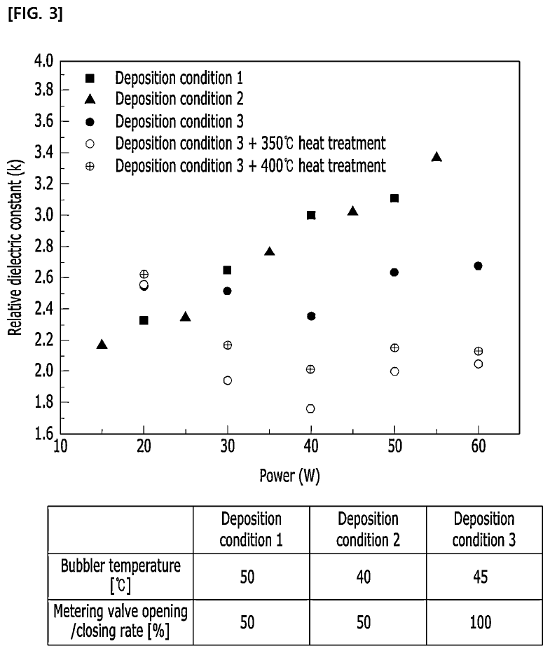

FIG. 3 is a graph showing relative dielectric constant values of a plasma polymer thin film (ppNP thin film) deposited at various power values using a first precursor material prepared according to one embodiment of the present application. According to this, the relative dielectric constant values varied from 1.78 to 3.38 depending on the conditions in which the ppNP thin film was deposited.

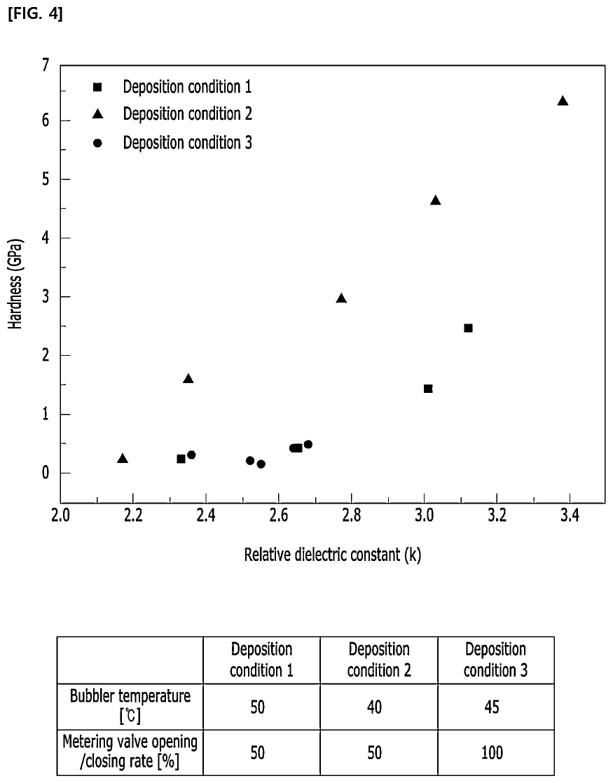

FIG. 4 is a graph showing hardnesses of a plasma polymer thin film (ppNP thin film) deposited using a first precursor material prepared according to one embodiment of the present application. Referring to FIG. 4, the hardnesses of the ppNP thin film increased from 0.4 GPa to 2.5 GPa as the relative dielectric constant increased.

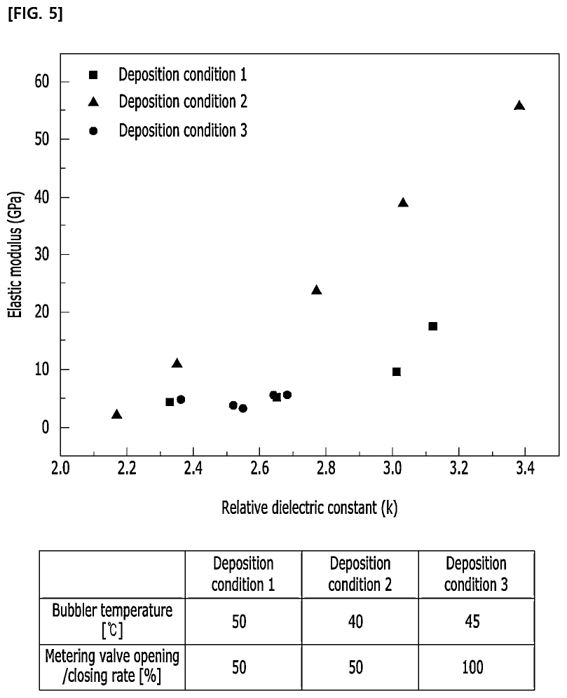

FIG. 5 is a graph showing elastic moduli (moduli) of a plasma polymer thin film (ppNP thin film) deposited using a first precursor material prepared according to one embodiment of the present application. Referring to FIG. 5, the elastic moduli of the ppNP thin film increased from 5 GPa to 18 GPa as the relative dielectric constant increased.

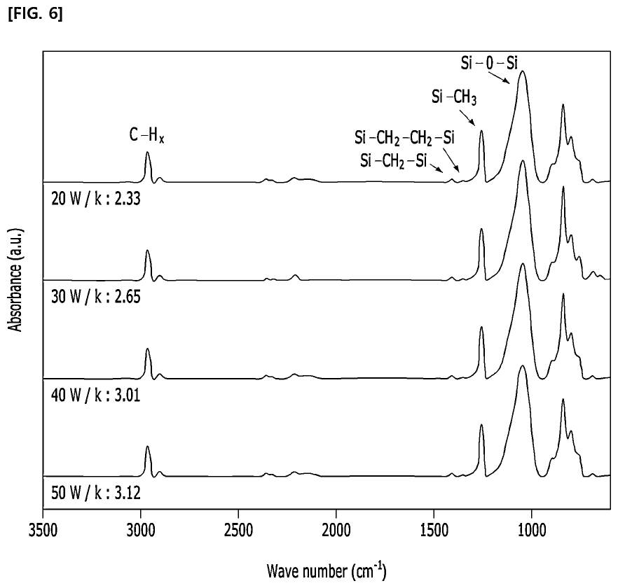

FIG. 6 is a graph showing chemical structures obtained by Fourier infrared spectroscopy in a plasma polymer thin film deposited using a first precursor material prepared according to one embodiment of the present application. Specifically, it is a graph showing the chemical structures of the ppNP thin film according to applied power. As shown here, it can be seen that the ppNP thin film basically occupies a plurality of Si—O—Si bonding structures and Si—(CH₃)ₓ bonding structures. In particular, as the power increased, the C—Hₓ bonding structure, the Si—CH₃ bonding structure, and the Si—(CH₃)ₓ bonding structure decreased, whereas a pattern of maintaining the Si—O—Si bond structure was shown. C—Hₓ bonding structure, Si—CH₃ bonding structure, and Si—(CH₃)ₓ bonding structure can lead to relatively lower dielectric constant, and Si—O—Si bonding structure can lead to stronger mechanical strength. Therefore, as shown in FIG. 3, the dielectric constant increased as the plasma power increased, and as the dielectric constant increased as shown in FIGS. 4 and 5, the mechanical strength, that is, the hardness and elastic modulus of the thin film were shown to be excellent.

In addition, mechanical strength superior to carbon bond structures such as Si—CH₃ was formed by forming Si—CH₂—Si and Si—CH₂—CH₂—Si structures derived from the Si—H bond of the present precursor.

### EXPERIMENTAL EXAMPLE 2

### Comparative Experiment of Example and Comparative Examples

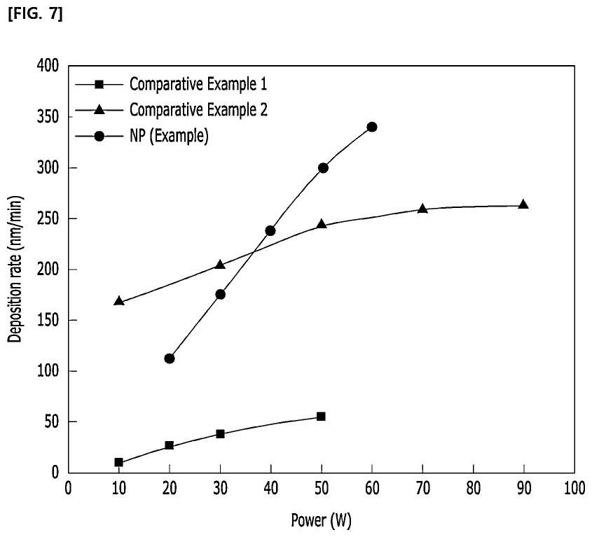

FIG. 7 is a graph showing deposition rates of plasma polymer thin films prepared according to an Example and Comparative Examples of the present application.

Referring to FIG. 7, it can be confirmed at a power of 40 W or more that the thin film according to Example of the present disclosure prepared of a T-shaped precursor had more excellent deposition rate than the thin films of Comparative Examples 1 and 2 prepared of cross-shaped and H-shaped precursors.

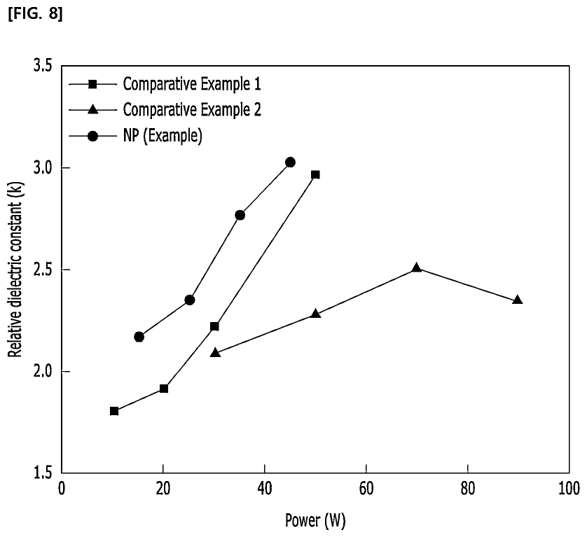

FIG. 8 is a graph showing relative dielectric constant values of plasma polymer thin films prepared according to an Example and Comparative Examples of the present application.

Referring to FIG. 8, it can be confirmed that the relative dielectric constant increased as the plasma power increased, and it can be confirmed that the change width after plasma exposure was smaller than those of Comparative Examples 1 and 2.

FIG. 9 is a graph showing relative dielectric constant values before and after heat treatment of plasma polymer thin films prepared according to an Example and Comparative Examples of the present application.

Referring to FIG. 9, it can be confirmed that the relative dielectric constant value of each thin film decreased after heat treatment.

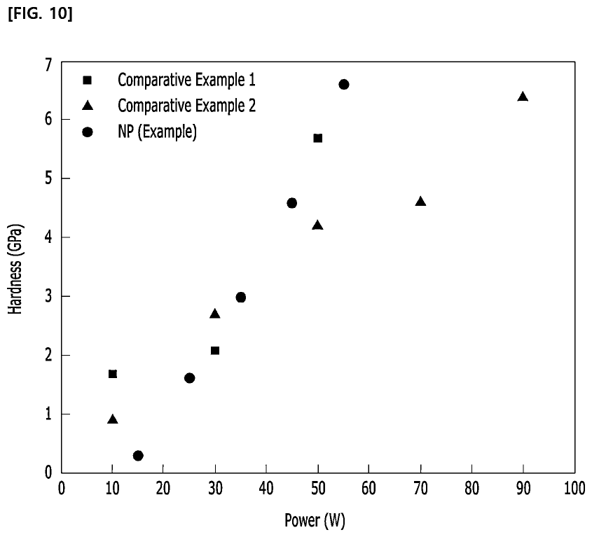

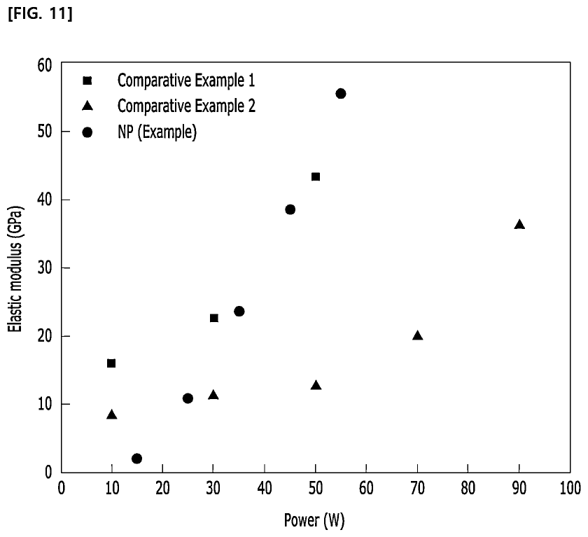

FIG. 10 is a graph comparing hardnesses of plasma polymer thin films prepared according to an Example and Comparative Examples of the present application, and FIG. 11 is a graph comparing elastic moduli of plasma polymer thin films prepared according to an Example and Comparative Examples of the present application.

Referring to FIGS. 10 and 11, it can be confirmed that the hardnesses and elastic moduli increased as the plasma power increased, and it can be confirmed that the increase width in Example was greater than those of Comparative Examples 1 and 2.

### EXPERIMENTAL EXAMPLE 3

### Comparative Experiment of Example and Comparative Examples

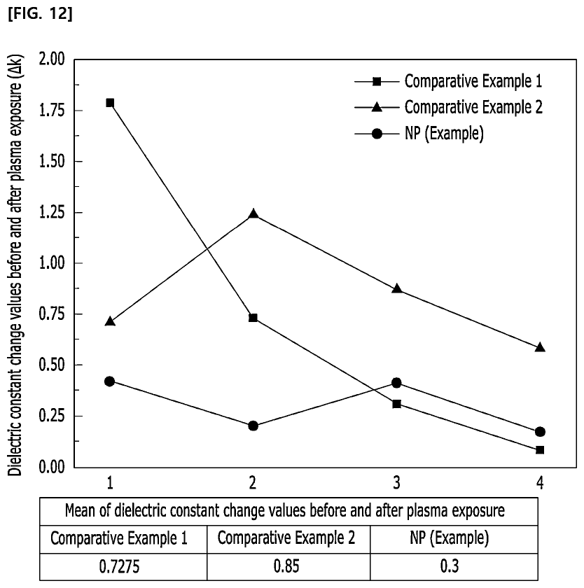

FIG. 12 is a graph showing relative dielectric constant values before and after plasma treatment of plasma polymer thin films prepared according to an Example and Comparative Examples of the present application.

Referring to FIG. 12, it can be confirmed that the thin film according to Example of the present disclosure prepared of a T-shaped precursor had a smaller change width in dielectric constant value after plasma exposure than the thin films of Comparative Examples 1 and 2 prepared of cross-shaped and H-shaped precursors.

### EXPERIMENTAL EXAMPLE 4

### Comparison of Characteristics according to Carbon Ratios

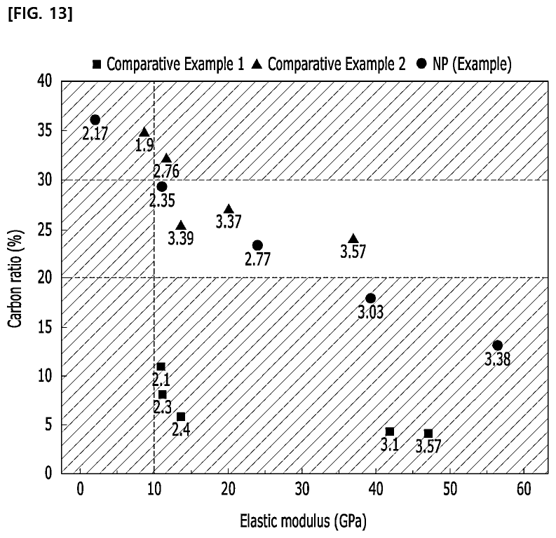

FIG. 13 is a graph showing dielectric constant values according to elastic moduli and carbon ratios of plasma polymer thin films prepared according to an Example and Comparative Examples of the present application.

Referring to FIG. 13, it can be confirmed that the thin films had low dielectric constant values compared to the mechanical strength in the region where the carbon composition ratios of the thin films were 20% to 30%, and in the corresponding region, the thin film according to Example of the present disclosure had the smallest change in dielectric constant values before and after plasma exposure.

The plasma polymer thin film is advantageous in forming a low dielectric constant value when the carbon ratio exceeds 30%, but there exists a problem in that the thin film is very soft and has poor elasticity. Meanwhile, at a carbon ratio of 20% to 15% or less, the plasma polymer thin film is advantageous for forming a low dielectric constant value and forming high elasticity, but there is a problem in that the characteristic change and the degree of damage to the thin film after plasma exposure increase.

Considering the above matters, the T-shaped precursor according to the present disclosure shows the most excellent characteristics in the region of a carbon ratio of 20% to 30%, which is less changed even after plasma exposure in the current semiconductor process than the cross-shaped and H-shaped precursors, which are the previous patents of the inventors of the present application.

### EXPERIMENTAL EXAMPLE 5

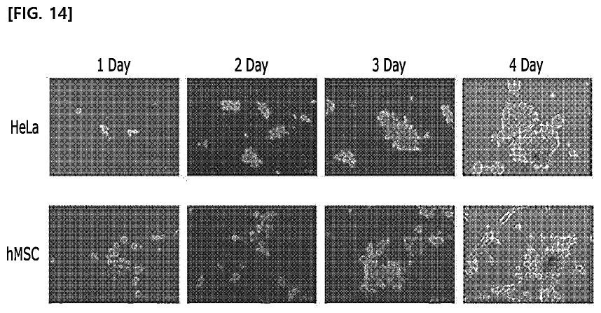

FIG. 14 is results of culturing stem cells in a plasma polymer thin film according to one embodiment of the present application.

Referring to FIG. 14, it can be confirmed that when HeLa cells and human mesenchymal stem (hMSC) cells were cultured, stem cells in the form of spheroids, which were not produced before coating, were produced.

The foregoing description of the present application is for illustration, and those with ordinary skill in the art to which the present application pertains will be able to understand that it can be easily modified into other specific forms without changing the technical spirit or essential features of the present application. Therefore, it should be understood that the embodiments described above are illustrative in all respects and not restrictive. For example, each element described as a single form may be implemented in a dispersed form, and likewise elements described in the dispersed form may also be implemented in a combined form.

The scope of the present application is indicated by the claims to be described later rather than the above detailed description, and all changes or modified forms derived from the meaning and scope of the claims and equivalent concepts thereof should be construed as being included in the scope of the present application.

What is claimed is:

1. A plasma polymer thin film prepared using a first precursor material represented by the following Chemical Formula 1: 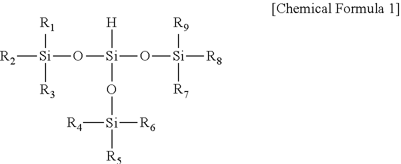
(in Chemical Formula 1,
R₁ to R₉ are each independently H or a C₁-C₅ substituted or unsubstituted alkyl group, and when R₁ to R₉ are substituted, the substituent is an amino group, a hydroxyl group, a cyano group, a halogen group, a nitro group, or a methoxy group).

2. The plasma polymer thin film of claim 1, wherein the plasma polymer thin film is prepared by using a second precursor material, which is a hydrocarbon of a liquid state at 25° C. and 1 atm, together with the first precursor material.

3. The plasma polymer thin film of claim 2, wherein the second precursor material includes C₆-C₁₂ alkane, alkene, cycloalkane, or cycloalkene.

4. The plasma polymer thin film of claim 2, wherein the second precursor material includes cyclohexane.

5. The plasma polymer thin film of claim 1, wherein the first precursor material has a T-shaped structure.

6. The plasma polymer thin film of claim 1, wherein the plasma polymer thin film is prepared using a plasma enhanced CVD (PECVD) method.

7. A method for preparing a plasma polymer thin film, wherein a plasma-polymerized thin film is deposited on a substrate using a first precursor material represented by the following Chemical Formula 1: 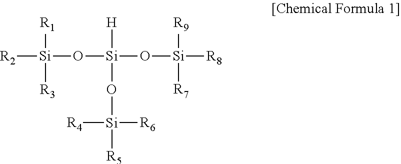
(in Chemical Formula 1,
R₁ to R₉ are each independently H or a C₁-C₅ substituted or unsubstituted alkyl group, and when R₁ to R₉ are substituted, the substituent is an amino group, a hydroxyl group, a cyano group, a halogen group, a nitro group, or a methoxy group).

8. The method of claim 7, wherein the step of depositing the plasma-polymerized thin film on the substrate is to deposit the plasma-polymerized thin film on the substrate by using a second precursor material, which is a hydrocarbon of a liquid state at 25° C. and 1 atm, together with the first precursor material.

9. The method of claim 8, wherein the second precursor material includes C₆-C₁₂ alkane, alkene, cycloalkane, or cycloalkene.

10. The method of claim 8, wherein the second precursor material includes cyclohexane.

11. The method of claim 7, further comprising a step of post-treating the thin film deposited on the substrate.

12. The method of claim 11, wherein the post-treatment is performed by a process selected from the group consisting of inductively coupled plasma (ICP) treatment, rapid thermal annealing (RTA), and a combination thereof.

13. The method of claim 7, wherein the step of depositing the plasma-polymerized thin film on the substrate includes steps of:
evaporating the first precursor material and the second precursor material in a bubbler;
inflowing the evaporated precursor materials from the bubbler to flow them into a reactor for plasma deposition; and
forming the plasma-polymerized thin film on the substrate within the reactor using plasma of the reactor.

14. The method of claim 13, wherein the reactor contains a carrier gas selected from the group consisting of argon (Ar), helium (He), neon (Ne), and combinations thereof.

15. The method of claim 13, wherein the reactor has a carrier gas pressure of 1×10⁻¹ Torr to 100×10⁻¹ Torr.

16. The method of claim 13, wherein the substrate within the reactor has a temperature of 20° C. to 50° C.

17. The method of claim 13, wherein the reactor has a power supplied thereto of 10 W to 90 W.

18. A stem cell culture substrate comprising the plasma polymer thin film according to any one of claims 1.

19. The stem cell culture substrate of claim 18, wherein the stem cells have a spheroid shape.
